(19) United States
(12) Patent Application Publication
(10) Pub. No.: US 2025/0339686 A1
(43) Pub. Date: Nov. 6, 2025

(54) ELECTROCOCHLEOGRAPHY-BASED INSERTION MONITORING

(71) Applicant: Cochlear Limited, Macquarie University, NSW (AU)

(72) Inventors: Zachary Mark Smith, Pymble, NSW (AU); Joerg Pesch, Mechelen (BE)

(21) Appl. No.: 18/860,753

(22) PCT Filed: Apr. 26, 2023
(86) PCT No.: PCT/IB2023/054309

§ 371 Date: Oct. 28, 2024

Related U.S. Application Data
(60) Provisional application No. 63/337,314, filed on May 2, 2022.

(51) Int. Cl. A61N 1/36; A61N 1/372

(52) U.S. Cl. CPC A61N 1/36039; A61N 1/37247

(57) ABSTRACT

Techniques presented herein disambiguate Electrocochleography (ECochG) signal changes caused by either a moving electrode contact or an underlying shift in the change in the acoustic responsiveness of the cochlea by contemporaneously recording ECochG signals from at least two sites in the cochlea and tracking the position or movement of the stimulating assembly during surgery. The recorded ECochG signals, along with the position information. can be used to determine the cause of variations in the measured ECochG signals.

### BACKGROUND

### Field of the Invention

The present invention relates generally to monitoring Electrocochleography (ECochG) signals during insertion of a stimulating assembly into a recipient.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method for insertion of a stimulating assembly comprising a plurality of electrode contacts into an inner ear of a recipient is provided. The method comprises: iteratively recording, over a period of time, a first Electrocochleography (ECochG) signal from a primary recording site: obtaining position information for the primary recording site in association with recordings of the first ECochG signal: and analyzing the first ECochG signal using at least the position information for the primary recording site.

In another aspect, a method is provided. The method comprises: iteratively delivering at least one acoustic stimulus to an inner ear of a recipient during insertion of a stimulating assembly into the inner ear, wherein the stimulating assembly comprises a plurality of electrode contacts: recording, at a primary recording site, a first Electrocochleography (ECochG) signal evoked in response delivery of the at least one acoustic stimulus: recording position information of one or more parts of the stimulating assembly during monitoring of the first ECochG signal: and monitoring an acoustic responsiveness of the inner ear based on at least the first ECochG signal and the position information.

In another aspect, one or more non-transitory computer readable storage media are provided. The one or more non-transitory computer readable storage media comprise instructions that, when executed by a processor, cause the processor to: obtain a second Electrocochleography (ECochG) signal iteratively recorded via an apical electrode of a stimulating assembly during insertion of the stimulating assembly into an inner ear of a recipient: obtain a first ECochG signal iteratively recorded via at least one other electrode of the stimulating assembly during insertion of the stimulating assembly into the inner ear; and analyze the second ECochG signal recorded via the apical electrode relative to the first ECochG signal recorded via at least one other electrode to characterize an acoustic responsiveness of the inner ear.

In another aspect, a system is provided. The system comprises: a user interface: a network interface for communication with an implantable medical device comprising a plurality of electrode contacts configured to be implanted into a recipient: a memory; and one or more processors configured to: obtain recordings of a first Electrocochleography (ECochG) signal from a primary recording site during implantation of the plurality of electrode contacts into the recipient: determine position information for the primary recording site in association with the recordings of the first ECochG signal: and analyze the recordings of the first ECochG signal using at least the position information.

### BRIEF DESCRIPTION OF THE DRAWINGS

Embodiments of the present invention are described herein in conjunction with the accompanying drawings, in which:

FIG. 1A is a graph illustrating a cochlear microphonic magnitude associated with an Electrocochleography (ECochG) signal captured during insertion of a stimulating assembly into an inner ear of a recipient;

FIG. 1B is a graph illustrating a cochlear microphonic magnitude associated with an ECochG signal captured during insertion of a stimulating assembly into another inner ear of a recipient;

FIG. 2 is a schematic diagram illustrating a cochlear implant system implanted in a head of a recipient and an ECochG insertion monitoring system associated with the cochlear implant system, in accordance with certain embodiments presented herein;

FIG. 3 is a schematic diagram illustrating the cochlear implant system and the ECochG insertion monitoring system of FIG. 2, showing a side view of the head of the recipient, in accordance with certain embodiments presented herein;

FIG. 4 is a schematic diagram illustrating the cochlear implant system and the ECochG insertion monitoring system of FIG. 2, showing components of the cochlear implant system without the recipient's head for purposes of clarity, in accordance with certain embodiments presented herein;

FIG. 5 is a block diagram of the cochlear implant system and the ECochG insertion monitoring system of FIG. 2, in accordance with certain embodiments presented herein;

FIGS. 6A, 6B, 6C, 6D, 6E, and 6F are a series of diagrams schematically illustrating recording of ECochG signals during insertion of a stimulating assembly into the inner ear of a recipient, in accordance with certain embodiments presented herein;

FIG. 7A is a graph illustrating cochlear microphonic magnitudes associated with ECochG signals recorded during insertion of a stimulating assembly into the inner ear of a recipient, in accordance with certain embodiments presented herein;

FIG. 7B is a graph illustrating angular insertion depth of electrode contacts during insertion of the stimulating assembly of FIG. 7A, in accordance with certain embodiments presented herein;

FIG. 7C is a zoomed in (enlarged view of the cochlear microphonic magnitude of FIG. 7A at a fixed target position within the inner ear, in accordance with certain embodiments presented herein;

FIG. 8A is a graph illustrating cochlear microphonic magnitudes associated with ECochG signals recorded during insertion of a stimulating assembly into the inner ear of a recipient, in accordance with certain embodiments presented herein;

FIG. 8B is a graph illustrating angular insertion depth of electrode contacts during insertion of the stimulating assembly of FIG. 8A, in accordance with certain embodiments presented herein;

FIG. 8C is a zoomed in (enlarged) view of the cochlear microphonic magnitude of FIG. 7A at a fixed target position within the inner ear, in accordance with certain embodiments presented herein;

FIG. 9A is a graph illustrating cochlear microphonic magnitudes associated with ECochG signals recorded during insertion of a stimulating assembly into the inner ear of a recipient, in accordance with certain embodiments presented herein;

FIG. 9B is a graph illustrating angular insertion depth of electrode contacts during insertion of the stimulating assembly of FIG. 9A, in accordance with certain embodiments presented herein;

FIG. 9C is a graph illustrating a ratio change of cochlear microphonic magnitudes recorded from a primary recording site in FIG. 9A relative to a cochlear microphonic magnitude previously recorded from a secondary recording site at the same cochlear location, in accordance with certain embodiments presented herein;

FIG. 10A is a graph illustrating cochlear microphonic magnitudes associated with ECochG signals recorded during insertion of a stimulating assembly into the inner ear of a recipient, in accordance with certain embodiments presented herein;

FIG. 10B is a graph illustrating angular insertion depth of electrode contacts during insertion of the stimulating assembly of FIG. 10A, in accordance with certain embodiments presented herein;

FIG. 10C is a graph illustrating a ratio change of cochlear microphonic magnitudes recorded from a primary recording site in FIG. 10A relative to a cochlear microphonic magnitude previously recorded from a secondary recording site at the same cochlear location, in accordance with certain embodiments presented herein;

FIG. 11 is a cross-sectional view of a cochlea and stimulating assembly illustrating a position of the stimulating assembly within the cochlea, in accordance with certain embodiments presented herein;

FIG. 12 is a schematic diagram illustrating a vestibular stimulator system and an ECochG insertion monitoring system associated with the vestibular stimulator system, in accordance with certain embodiments presented herein;

FIG. 13 is a flowchart of a method, in accordance with certain embodiments presented herein; and

FIG. 14 is a flowchart of another method, in accordance with certain embodiments presented herein.

### DETAILED DESCRIPTION

Auditory/hearing prosthesis recipients suffer from different types of hearing loss (e.g., conductive and/or sensorineural) and/or different degrees/severity of hearing loss. However, it is now common for many hearing prosthesis recipients to retain some residual natural hearing ability (residual hearing) after receiving the hearing prosthesis. That is, hearing prosthesis recipients often retain at least some of their natural ability to hear sounds without the aid of their hearing prosthesis. For example, cochlear implants can now be implanted in a manner that preserves at least some of the recipient's cochlear hair cells and the natural cochlear function, particularly in the lower frequency regions of the cochlea.

Electrocochleography (ECoG or ECochG) refers to a clinical measurement technique that can be used to, for example, assess a recipient's residual hearing. ECochG involves the delivery of acoustic stimuli to a recipient's cochlea, and recording one or more responses (ECochG responses or ECochG signals) of the cochlea to the acoustic stimulus. For example, during certain ECochG testing procedures, preselected/predetermined clicks or tones are delivered acoustically to the inner ear of recipient and an ECochG response/signal is recording, for example, using an electrode in or near the patient's middle ear or inner ear.

ECochG recording can be used during insertion of a stimulating assembly into the cochlea. For example, conventional arrangements record an ECochG signal from the most apical electrode and monitor the amplitude and latency of the cochlear microphonic (CM). Drops in the cochlear microphonic amplitude and/or sudden changes in latency are typically interpreted to mean that something went wrong, and, in some cases, surgeons will retract, or otherwise manipulate the position of, the stimulating in an attempt to recover the cochlear microphonic.

However, it is difficult for a user (e.g., surgeon) to determine if observed changes in the ECochG signal (e.g., cochlear microphonic drops and jumps) are indicative of the recording electrode passing by different local patterns of cochlear anatomy (e.g., due to outer hair cell (OHC) health) or a change in the acoustic responsiveness of the cochlea (e.g., changed contact with basilar membrane, trauma, etc.). This makes it very difficult to interpret ECochG signal changes during cochlear implant surgery beyond equating all drops in amplitude to a problem, especially when creating an automated ECochG interpretation system.

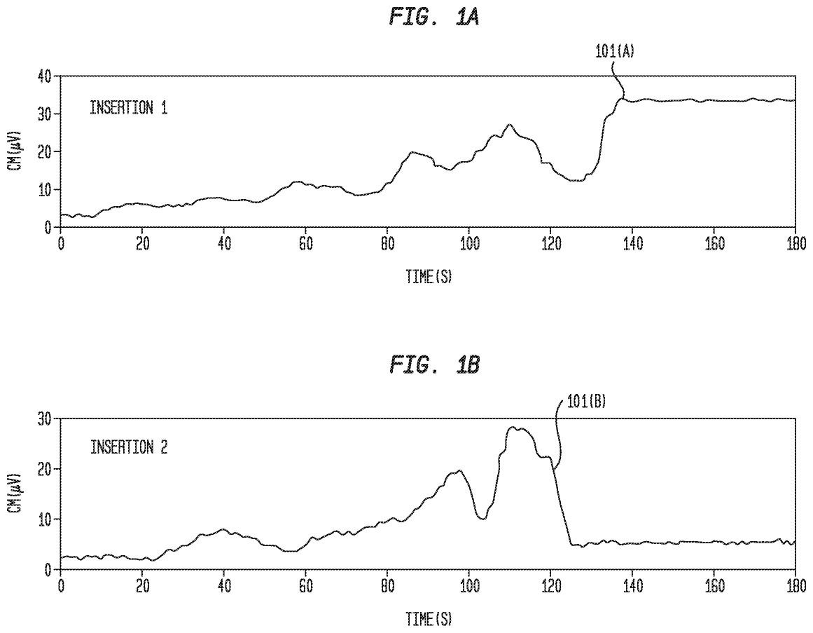

For example, FIG. 1A is a graph illustrating part of an ECochG signal. namely a cochlear microphonic (CM) magnitude 101(A), as a function of time for the surgical insertion of a first stimulating assembly into a first cochlea. FIG. 1B is a graph illustrating a cochlear microphonic magnitude 101(B), as a function of time for the surgical insertion of a second stimulating assembly into a second cochlea. The cochlear microphonic magnitudes 101(A) and 101(B) shown in FIGS. 1A and 1B, respectively. represent recordings made from the most apical electrode contact for a 500 Hertz (Hz) probe stimulus. Note that in both cases there are several rises and falls in the cochlear microphonic magnitude over the course of the insertions and that it is difficult to determine the cause of each drop in magnitude from these recordings alone.

The techniques presented herein operate to disambiguate ECochG signal changes caused by either (1) a moving electrode contact or (2) an underlying shift in the acoustic responsiveness of the cochlea by contemporaneously recording ECochG signals from at least two sites in the cochlea and tracking the position or movement of the stimulating assembly during surgery. This enables comparison of the ECochG signals at different time points from substantially the same, or very similar, location (e.g., same tonotopic frequency region) with changes in overall insertion depth of the stimulating assembly. The position or relative movement, of the stimulating assembly can be tracked by impedance monitoring of the electrode array contact, analysis of the phase/latency of the ECochG signal, visual tracking (surgical microscope), or radiographic video imaging such as fluoroscopy. The recorded ECochG signals, along with the position information, can be used to determine the cause of variations in the measured ECochG signals, which are not easily determined in the current state of the art. By improving the interpretation of recorded ECochG signals, surgeons should be able to preserve hearing during electrode insertion with more confidence.

As used herein, the “position” of an electrode contact generally refers to the insertion depth (e.g., angular insertion depth) of the electrode contact in the inner ear (e.g., cochlea). However, the “position” of the electrode contact can also include the relative proximity of the electrode contact to a wall of the inner ear (e.g., modiolar proximity, lateral wall proximity, etc.), distance from the mid-modiolar axis, or other information relating to the placement or position of one or more parts of the stimulating assembly.

In general, the techniques presented herein record ECochG signals and electrode contact position information, and then analyze this information to determine whether detected ECochG signal changes are due to local anatomy variations as the stimulating assembly moves through the inner ear (e.g., not requiring surgical intervention/remediation), or whether the detected ECochG signal changes are due to a change in the acoustic responsiveness of the cochlea (e.g., requiring surgical intervention/remediation). In accordance with certain embodiments presented herein, a system iteratively records (e.g., continuously, periodically, etc.) ECochG signals from a “primary recording site” and, potentially, “secondary recording site” within the inner ear (e.g., cochlea). In these embodiments, the stimulating assembly comprises an elongate carrier member having a plurality of longitudinally spaced electrode contacts. As described further below, different options for iteratively recording ECochG signals during insertion of a stimulating assembly into the inner ear, and for analyzing the ECochG signals with electrode contact position information, are presented herein.

In certain embodiments, the secondary recording site is fixed to the most apical electrode contact of the stimulating assembly, meaning that the position/location (e.g., insertion depth, modiolar proximity, etc.) of the secondary recording site will change over time as the stimulating assembly is progressively inserted into the inner ear or otherwise manipulated, but that the electrode used to make the secondary recording does not change (i.e., the secondary recording site is the most apical electrode). However, in these embodiments, the one or more primary recording sites within the inner ear are at fixed positions (e.g., predetermined insertion depth), meaning that the position/location of the one or more secondary sites will remain substantially constant/fixed over time as the stimulating assembly is progressively inserted into the inner ear or otherwise manipulated, but that the electrode contact(s) used to make the secondary recording(s) will change over time (e.g., electrode changes to be the electrode most proximate a predetermined constant position within the inner ear). The locations for the one or more primary recording sites can be, for example, near the base of the cochlea or another fixed location with a robust ECochG response (e.g., will not record at the cochlea base if the ECochG signal is very small or absent).

In accordance with alternative embodiments, the secondary recording site and the one or more primary recording sites are each fixed to the a specific electrode contact of the stimulating assembly, meaning that the position/location (e.g., insertion depth, modiolar proximity, etc.) of the secondary recording site and the one or more primary recording sites will change over time as the stimulating assembly is progressively inserted into the inner ear or otherwise manipulated, but that the electrodes used to make the secondary recording and one or more secondary recordings do not change. The secondary recording site can be, for example, the most apical electrode and the one or more primary recording sites are each more basal electrodes (e.g., an electrode spaced some distance from the most apical electrode).

In accordance with these embodiments, for each ECochG signal recording, the inner car positions of the electrodes when the recording was made, and potentially the time at which the recording was made, are also obtained/recorded and associated with the corresponding ECochG signal recording. This information is then used by the system to analyze the ECochG signals in a relative manner to determine when and ECochG signal changes are due to local anatomy variations as the stimulating assembly moves through the inner ear, or whether the detected ECochG signal changes are due to a change in the acoustic responsiveness of the cochlea (e.g., compare the ECochG signal over time at one or more known positions to determine if ECochG changes observed at the most apical electrode are cause by local anatomy or a change in the acoustic responsiveness of the cochlea). As such, the techniques presented herein can provide more clear interpretations of ECochG signals and the resulting implications for surgeons, thereby leading to improved surgical interventions to maximize/balance hearing preservation and stimulating assembly insertion depth. The techniques presented herein also facilitate the creation of algorithms that automate the interpretation of ECochG signals and provide surgeons with more meaningful information to guide their decision process and maximize preservation of residual hearing and overall outcomes.

Merely for ease of description, the techniques presented herein are primarily described with reference to a specific implantable medical device system, namely a cochlear implant system. However, it is to be appreciated that the techniques presented herein can also be partially or fully implemented by other types of implantable medical devices, including other implantable devices that bave the ability to record ECochG signals For example, the techniques presented herein can be implemented by other auditory prosthesis systems that include one or more other types of auditory prostheses, such as middle ear auditory prostheses, bone conduction devices, direct acoustic stimulators, electro-acoustic prostheses, auditory brain stimulators, combinations or variations thereof, etc. The techniques presented herein can also be implemented by dedicated tinnitus therapy devices and tinnitus therapy device systems. In further embodiments, the presented herein can also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

As used herein, an ECochG signal can include one or a plurality of different stimulus related electrical potentials (e.g., a set of ECochG responses) that include the cochlear microphonic (CM), the cochlear summating potential (SP), and the auditory nerve neurophonic (ANN)/auditory nerve Action Potential (AP), where these parameters are measured/recorded independently or in various combinations in response to delivery of an acoustic stimulus to the inner ear. The cochlear microphonic is an alternating current (AC) voltage that mirrors the waveform of the acoustic stimulus at low to moderate levels of acoustic stimulation. The cochlear microphonic is generated by the outer hair cells of the organ of Corti and is dependent on the proximity of the recording electrode(s) to the stimulated hair cells. In general, the cochlear microphonic is proportional to the displacement of the basilar membrane.

The summating potential is the direct current (DC) response of the outer hair cells of the organ of Corti as they move in conjunction with the basilar membrane (i.e., reflects the time-displacement pattern of the cochlear partition in response to the stimulus envelope). The summating potential is the stimulus-related potential of the cochlea and can be seen as a DC (unidirectional) shift in the cochlear microphonic baseline. The direction of this shift (i.e., positive or negative) is dependent on a complex interaction between stimulus parameters and the location of the recording electrode(s).

The auditory nerve neurophonic (auditory nerve action potential) represents the summed response of the synchronous firing of the nerve fibers in response to the acoustic stimuli, and it appears as an alternating current voltage. The auditory nerve neurophonic is characterized by a series of brief, predominantly negative peaks, including a first negative peak (N1) and second negative peak (N2). The auditory nerve neurophonic also includes a magnitude and a latency. The magnitude of the auditory nerve neurophonic reflects the number of fibers that are firing, while the latency of the auditory nerve neurophonic is measured as the time between the onset and the first negative peak (N1). In general, the ECochG signal recording can be completed within a short time period (e.g., a few milliseconds after the initial delivery of the acoustic stimuli) and does not have to wait until after completion of the acoustic stimuli.

For ease of description, the techniques are primarily described herein with reference to analysis of the cochlear microphonic and, particularly, the cochlear microphonic magnitude at different inner ear positions. However, it is to be appreciated that specific reference to the cochlear microphonic magnitude is merely illustrative and that the techniques presented herein can be implemented with other parameters of the ECochG signal, including the summating potential, the auditory nerve neurophonic. The relative analysis of these parameters of the ECochG signal can include analysis of one or more of the parameter magnitudes, parameter latencies, etc.

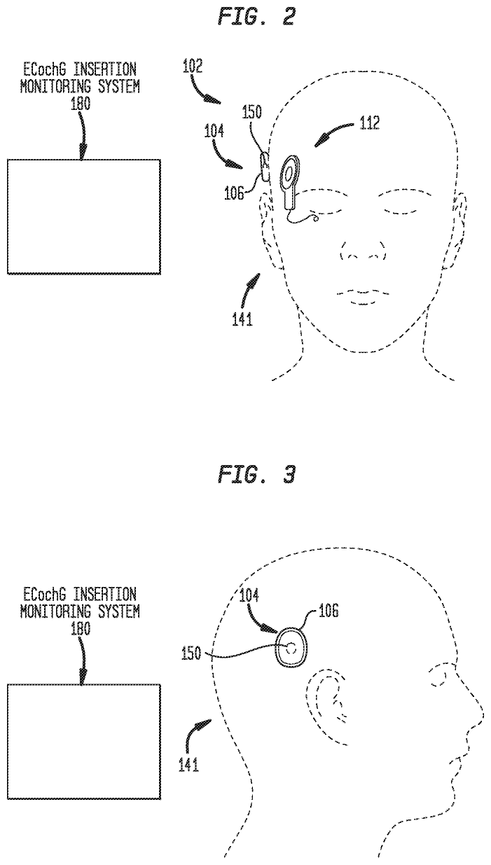

Referring initially to FIGS. 2-5, which are generally described together for ease of description, an example cochlear implant system 102 can be implanted in a head 141 of a person, animal, or other recipient (each referred to herein as a “recipient”). The cochlear implant system 102 includes an external component 104 and an implantable component 112. The implantable component 112 is sometimes referred to as a “cochlear implant.” The cochlear implant system 102 operates with an Electrocochleography (ECoG or ECochG) insertion monitoring system 180.

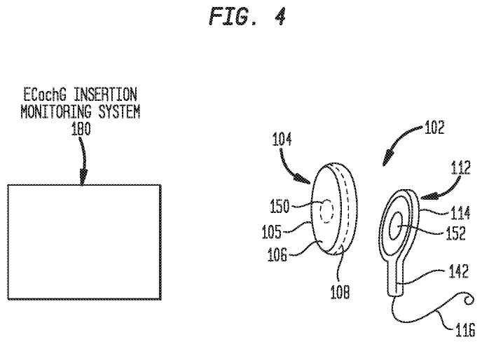

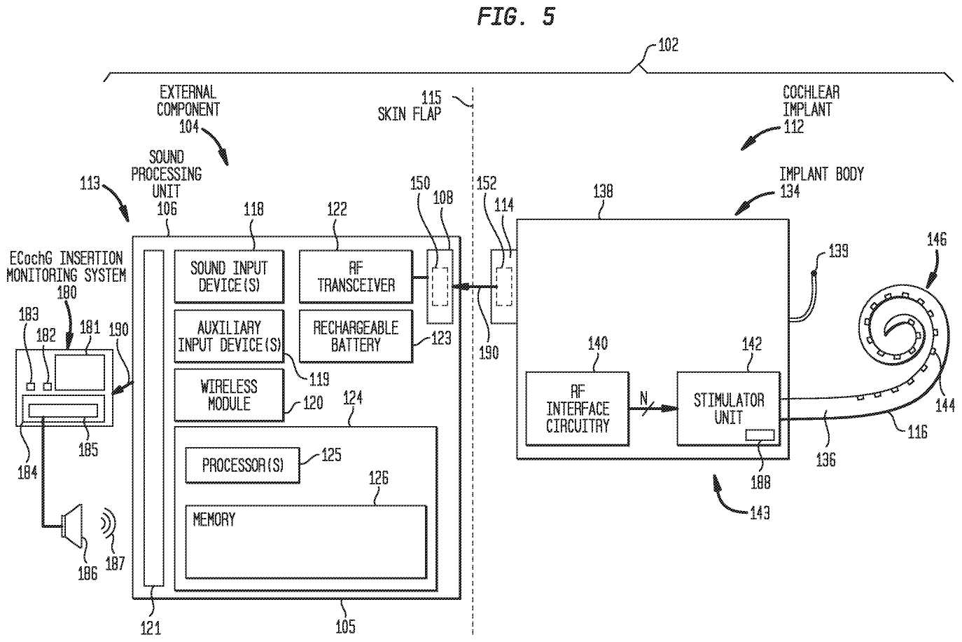

FIG. 2 is a schematic diagram illustrating the implantable component 112 implanted in the head 141 of the recipient, while FIG. 3 is a schematic diagram illustrating the external component 104 configured to be positioned adjacent the head 141 of the recipient. FIG. 4 includes another schematic view of the cochlear implant system 102, including both the external component 104 and the implantable component 112, but without the recipient's head being shown for purposes of clarity. FIG. 5 is a block diagram illustrating further details of the cochlear implant system 102 and the ECochG insertion monitoring system 180 associated with the cochlear implant system 102, in accordance with certain embodiments presented herein.

As noted, the cochlear implant system 102 includes an external component 104 that is configured to be directly or indirectly attached to the body of the recipient and an implantable component 112 configured to be implanted in the recipient. In the examples of FIGS. 1-4, the external component 104 comprises a sound processing unit 106, which is an off-the-ear (OTE) sound processing unit sometimes referred to as an “OTE component.” The sound processing unit 106 is configured to send data and power to the implantable component 112 as described below.

In the arrangement shown in FIGS. 2-5, the sound processing unit 106 includes a generally cylindrically shaped housing 105, which is configured to be magnetically coupled to the recipient's head 141. For example, the sound processing unit 106 can include an integrated external magnet 150 configured to be magnetically coupled to an implantable magnet 152 in the implantable component 112. The sound processing unit 106 also includes an integrated external coil 108 that is configured to be wirelessly (e.g., inductively) coupled to an implantable coil 114 of the implantable component 112 as described below. In FIGS. 1-3, the external magnet 150 is shown using dashed lines, indicating it is integrated within the housing 105 of the sound processing unit 106. In FIG. 5, the external magnet 150 and the implantable magnet 152 are shown using dashed lines, indicating the external coil 108 and the implantable coil 114 are disposed around the magnet 150 and magnet 152, respectively.

It is to be appreciated that the arrangement shown in FIGS. 2-5 is merely illustrative and that other arrangements are possible. In particular, the OTE sound processing unit 106 is merely illustrative of the external devices that could operate with the implantable component 112. For example, in alternative examples, the external component can comprise a behind-the-ear (BTE) sound processing unit or a micro-BTE sound processing unit and a separate external coil assembly. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

More generally, the OTE sound processing unit 106 is used for communication between the ECochG insertion monitoring system 180 and the cochlear implant 112. As such, during a surgical procedure, the OTE sound processing unit 106 could be replaced by any other device that is able to communicate with the ECochG insertion monitoring system 180 and the cochlear implant 112. In certain embodiment, the OTE sound processing unit 106 could be a so-called “surgical processor” having less capabilities than the OTE sound processing unit 106 (e.g., no sound processing logic, etc.). In various embodiments, the communication between the ECochG insertion monitoring system 180 and the OTE sound processing unit 106 or another device operating in place of the OTE sound processing unit 106, could communicate via a wireless or wired connection.

In addition, while FIGS. 2-5 illustrate an arrangement in which the cochlear implant system 102 includes an external component, it is to be appreciated that embodiments of the present invention can be implemented in cochlear implant systems having alternative arrangements. For example, embodiments presented herein can be implemented with a totally implantable cochlear implant or other totally implantable medical device. A totally implantable medical device is a device in which all components of the device are configured to be implanted under skin/tissue of a recipient. Because all components are implantable, a totally implantable medical device operates, for at least a finite period of time, without the need of an external device/component. However, an external component can be used to, for example, charge the internal power source (battery) of the totally implantable medical device.

Returning to the specific example of FIGS. 2-5 FIG. 5 illustrates that the sound processing unit 106 comprises one or more input devices 113 that are configured to receive input signals (e.g., sound or data signals). The one or more input devices 113 include one or more sound input devices 118 (e.g., microphones, audio input ports, telecoils, etc.), one or more auxiliary input devices 119 (e.g., audio ports, such as a Direct Audio Input (DAI), data ports, such as a Universal Serial Bus (USB) port, cable port, etc.), and a wireless transmitter/receiver (transceiver) 120. However, it is to be appreciated that the one or more input devices 113 can include additional types of input devices and/or less input devices (e.g., the wireless transceiver 120 and/or one or more auxiliary input devices 119 could be omitted).

The sound processing unit 106 also comprises the external coil 108, a charging coil 121, closely-coupled interface circuitry (transceiver) 122, sometimes referred to as a radio-frequency (RF) interface circuitry 122, at least one rechargeable battery 123, and a processing module 124. The processing module 124 comprises one or more processors 125 and a memory device (memory) 126. The memory device 126 can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors 125 are, for example, microprocessors or microcontrollers.

The implantable component 112 comprises an implant body (main module) 134, a lead region 136, and an intra-cochlear stimulating assembly 116, all configured to be implanted under a skin/tissue of the recipient. The magnets 150 and 152 magnetically couple the external component 104 to the implantable component 112 through the skin/tissue. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the transceiver 140 via a hermetic feedthrough (not shown in FIG. 5).

The stimulating assembly 116 is configured to be at least partially implanted in the recipient's cochlea. The stimulating assembly 116 includes a plurality of longitudinally spaced intra-cochlear electrical stimulating contacts (electrodes) 144 that collectively form a contact or electrode array 146 for delivery of electrical stimulation (current) to the recipient's cochlea. The stimulating assembly 116 extends through an opening in the recipient's cochlea (e.g., cochleostomy, the round window, etc.) and has a proximal end connected to stimulator unit 142 via the lead region 136 and hermetic feedthrough. The lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. In certain example embodiments, the external magnet 150 is fixed relative to the external coil 108, and the implantable magnet 152 is fixed relative to the implantable coil 114. The magnets 150 and 152 can facilitate operational alignment of the external coil 108 with the implantable coil 114 thereby enabling the external component 104 to transmit data and power to the implantable component 112 via a closely-coupled wireless link formed between the coils 108 and 114. In certain examples, the closely-coupled wireless link is a radio frequency (RF) link. However, various other types of energy transfer, such as infrared (IR), electromagnetic, capacitive, and inductive transfer can be used to transfer the power and/or data from an external component to an implantable component and, as such, FIGS. 2-5 illustrate only one example arrangement. For example, the external coil 108 can be in electrical communication with a power supply (e.g., the rechargeable battery 123) and can induce a current in the implantable coil 114, via an inductive link between the coils 108 and 114, to supply power to the implantable component 112.

The ECochG insertion monitoring system 180 includes, among other elements, a user interface 181, one or more processors 182, a network interface (e.g., wireless module) 183, and a memory device (memory) 184 storing ECochG insertion monitoring logic 185. The memory device 184 can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors 182 are, for example, microprocessors or microcontrollers configured to execute instructions associated with the ECochG insertion monitoring logic 185.

The network interface 183 enables communication with the external component 104 and/or the cochlear implant 112. For example, the network interface 183 can comprise a wireless module that is similar to wireless module 120, described elsewhere herein, for wireless communication with the external component 104 (or cochlear implant 112, if enabled with a wireless module).

The user interface 181 comprises, for example, one or more input devices over which the ECochG insertion monitoring system 180 receives input from a user, and one or more output devices by which the ECochG insertion monitoring system 180 is able to provide output to a user. The one or more input devices can include physically-actuatable user-interface elements (e.g., buttons, switches, or dials), touch screens, keyboards, mice, pens, and voice input devices, among others input devices. The one or more output devices can include, displays, speakers, and printers, among other output devices.

The ECochG insertion monitoring system 180 could be implemented by an suitable computing system, environment, or configuration including, but are not limited to, personal computers, server computers, hand-held devices, laptop devices, multiprocessor systems, microprocessor-based systems, programmable consumer electronics (e.g., smart phones), network PCs, minicomputers, mainframe computers, tablets, distributed computing environments that include any of the above systems or devices, and the like.

In accordance with embodiments presented herein, the ECochG insertion monitoring system 180 is configured to record ECochG signals from a primary recording site and, potentially, one or more secondary sites as the stimulating assembly 116 is inserted into the recipient's cochlea. More specifically, the ECochG insertion monitoring system 180 is configured to use electrodes 144 of the electrode array 146 to capture ECochG signals from the cochlea.

In a normal or fully functional ear, an acoustic pressure or sound wave (i.e., a sound signal) is collected by the outer ear and channeled into and through the ear canal. Disposed across the distal end of ear cannel is a tympanic membrane that vibrates in response to sound wave. This vibration is coupled to the oval window through three bones of middle ear. The middle ear bones serve to filter and amplify sound wave, causing the oval window to articulate, or vibrate, in response to vibration of tympanic membrane. This vibration sets up waves of fluid motion of the perilymph within the cochlea to active the cochlea hair cells. Activation of the hair cells causes appropriate nerve impulses to be generated and transferred through the recipient's spiral ganglion cells and auditory nerve to the brain where they are perceived as sound.

As noted above, it is common for hearing prosthesis recipient's to retain at least part of this normal hearing functionality (i.e., retain at least one residual hearing). Therefore, the cochlea of hearing prosthesis recipient can be acoustically stimulated upon delivery of a sound signal to the recipient's outer ear without the aid of the hearing prosthesis itself. In certain recipients, the normal hearing functionality can be enhanced through the use of an acoustic transducer in or near the outer ear and/or ear canal. In such recipients, the acoustic transducer is used to, for example, filter, enhance, and/or amplify a sound signal which is delivered to the cochlea via the middle ear bones and oval window, thereby creating waves of fluid motion of the perilymph within the cochlea. In other recipients, the normal hearing functionality can be enhanced through the use of a mechanical transducer that is coupled to the individual's bone (e.g., skull, jaw, etc.). In such recipients, the mechanical transducer delivers vibration to the individual's bone, and the vibration is relayed to the cochlea so as to create waves of fluid motion of the perilymph within the cochlea.

As such, an ECochG recording used in accordance with embodiments presented herein can be initiated by the ECochG insertion monitoring system 180. The ECochG recording involves the delivery of acoustic stimuli to the recipient's cochlea, and recording one or more responses of the cochlea to the acoustic stimulus. As used herein, acoustic stimuli refer to any type of stimulation that is delivered in a manner so as to set up waves of fluid motion of the perilymph within the cochlea that, in turn, activates the hair cells inside of cochlea. As such, acoustic stimuli for performance of an ECochG recording in accordance with embodiments presented herein can be delivered via a recipient's normal hearing functionality, via an acoustic transducer, via a mechanical transducer, a combination thereof, etc.

FIG. 5 illustrates an embodiment in which an acoustic transducer in the form of an external speaker 186 delivers acoustic stimulus 187 to the cochlea of the recipient. FIG. 5 also illustrates that the cochlear implant 112 includes a recording module 188 that is configured to record ECochG signals induced in the cochlea by the acoustic stimulus. The recording module 188 can comprise, for example, sense amplifiers configured to digitally record ECochG signals/responses presented on an input line connected to one or more of the electrodes 144. Data recorded by the sense amplifiers can, in certain embodiments, be stored in a buffer.

The RF interface circuitry 140 and 122 cooperate to provide ECochG signal data (e.g., the captured ECochG signals and data associated with captured ECochG signals, including recording site and time information) to the sound processing unit 106, where the ECochG signal data is then provided to the ECochG insertion monitoring system 180. The ECochG signal data is generally represented in FIG. 5 by arrows 190.

As noted above, presented herein are several options for recording and analyzing ECochG signals during insertion of stimulating assembly into the cochlea. These options are described below in greater detail below. For ease of illustration, the following description will also be, unless otherwise noted, explained with reference to the cochlear implant system 102 and ECochG insertion monitoring system 180 of FIGS. 2-5.

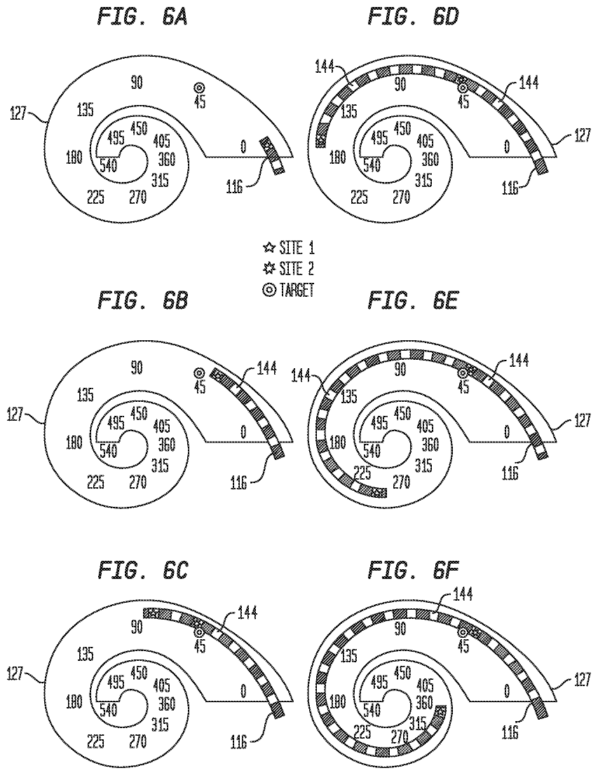

Referring first to FIGS. 6A-6F, shown are a series of diagrams illustrating advancement of stimulating assembly 116 into cochlea 127. Shown in each of FIGS. 6A-6F is the secondary recording site (represented by the 5-pointed star shape), a primary recording site (represented by the 7-pointed star shape), and a target position/location for the primary recording site (represented by the bulls-eye shape). In this example, the target position was chosen to be near an angular insertion depth of approximately 45°.

As shown in FIG. 6A, the stimulating assembly 116 is inserted into the cochlea 127 and the acoustic stimulus 187 (e.g., 500 Hertz (Hz) tone pip) is iteratively delivered to the cochlea 127. The recording module 188 records at least a few milliseconds of the voltage traces of the acoustic evoked response (ECochG signal) at the secondary recording site (i.e., the most apical electrode contact). This process continues iteratively until the stimulating assembly 116 is inserted, the surgeon pauses the insertion, or some other stop condition is reached. As shown, the secondary recording site is fixed with respect to the stimulating assembly 116 and moves with respect to the cochlea 127 as the stimulating assembly is advanced into the cochlea.

As the stimulating assembly 116 is inserted, the ECochG insertion monitoring system 180 iteratively (e.g., periodically, continuously, etc.) estimates the position of the secondary recording site (most apical electrode) relative to the cochlea 127 (e.g. determine the angular insertion depth for one or more electrode contacts inside the cochlea at least 2× per second, and optionally also determine the position of the electrode contacts within the cross-sectional plane of the cochlear turn at each angle, or in the cylindrical coordinate system, or other 3-dimensional coordinate system).

As noted, the ECochG insertion monitoring system 180 obtains (e.g., receives, determines, etc.) a target position in the cochlea 127 for use as the primary recording site. The ECochG insertion monitoring system 180 also records what is referred to herein as a “secondary recording reference” when the secondary site initially reaches the target position. That is, as used herein, the secondary recording reference is the ECochG signal (or parameter of the ECochG signal) recorded when the secondary recording site and the primary recording site are at the same cochlea position (e.g., when the most apical electrode reaches the predetermined target position).

As the stimulating assembly 116 is inserted, the most apical electrode will move past the target position, because, as noted, the primary recording site moves with respect to the stimulating assembly 116 and tries to maintain a fixed position with respect to the cochlea 127. Once a selected electrode other than the most apical electrode reaches the predetermined target position, the recording module 188 records at least a few milliseconds of the voltage traces of the acoustic evoked response (ECochG signal) at the selected electrode in response to the acoustic stimulus 187. That is, as noted, the acoustic stimulus 187 is delivered to the cochlea 127 and the ECochG signal evoked at the predetermined target position is recorded via the selected electrode. This means that the ECochG insertion monitoring system 180 records ECochG signals from at both the secondary recording site (most apical electrode) and the primary recording site (selected electrode located at the predetermined target position), either interleaved or simultaneously. Again, this process continues iteratively until the stimulating assembly 116 is inserted, the surgeon pauses the insertion, or some other stop condition is reached. However, since the primary recording site is fixed with respect to the cochlea 127, the primary recording site changed with respect to the stimulating assembly 116, meaning that different electrodes will be used as primary recording site as the insertion progresses. This use of different electrodes (i.e., progressively more basal electrodes) is schematically shown in FIGS. 6D, 6E, and 6F.

The predetermined target position can be selected, for example, by tracking the magnitude of the ECochG signal (acoustic evoked response) at the secondary recording site and the position of the secondary recording site in the cochlea 127 to determine when the magnitude of the ECochG signal first exceeds a threshold. Once this is detected, the position at which the ECochG signal first exceeds a threshold is set the target position for the primary recording site. Alternatively, the system could, a priori, set the target position.

In operation, based on the most recent positional estimate of the stimulating assembly 116, the ECochG insertion monitoring system 180 can continuously determine the nearest electrode contact to the target position, update the primary recording site to this electrode contact, and record at least a few milliseconds of the voltage trace of the acoustic evoked response (ECochG signal) at the primary recording site (nearest electrode contact to target position/location). If the stimulating assembly 116 has not moved significantly, the secondary recording site and primary recording site can use the same electrode contact. As the electrode is inserted deeper into the cochlea, the secondary recording site and primary recording site will be increasingly more distant from one another.

The ECochG insertion monitoring system 180 monitors the ECochG signal recorded from the secondary recording site based on the ECochG signal recording from the primary recording site. For example, the ECochG insertion monitoring system 180 compares the secondary recording reference (e.g., magnitude of the ECochG signal when the apical electrode was at the target position) with the most recent recording from the primary recording site. The system can, for example, plot the difference, or ratio, of the primary recording site magnitude compared to the secondary recording site magnitude from the target position. After optionally compensating for differences in position, if the primary recording site magnitude is smaller than the secondary recording reference, and the difference exceeds a threshold, for example 30% smaller, then the system can determine that a significant drop in the acoustic evoked response has occurred.

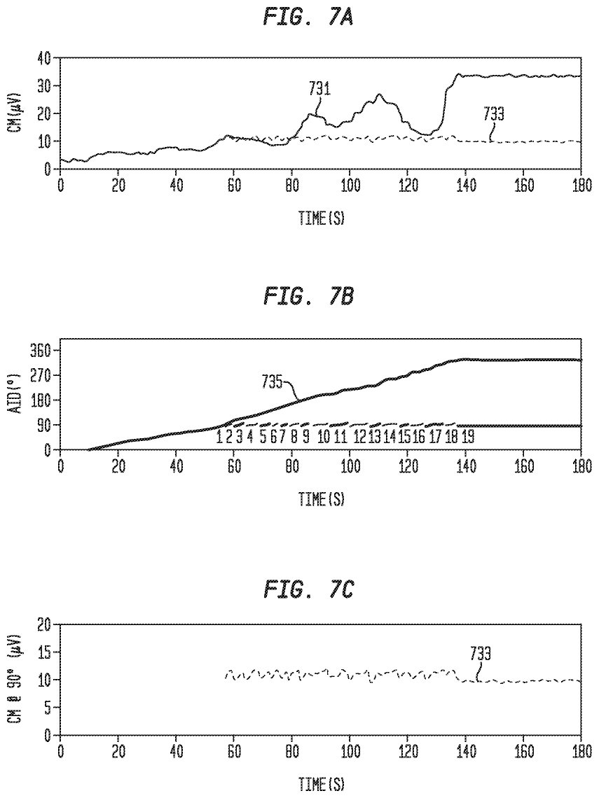

FIGS. 7A, 7B, and 7C schematically illustrate example monitored parameters of the ECochG signals obtained during one example insertion of stimulating assembly 116 into cochlea 127. However, whereas the target position for the primary recording site in FIGS. 6A-6F was an angular insertion depth of approximately 45°, in the examples of FIGS. 7A-7C the target position for the primary recording site is approximately 90°.

More specifically, FIG. 7A includes trace/line 731 representing the cochlear microphonic (CM) amplitude/magnitude recorded from the secondary recording site, i.e., the most apical electrode contact during insertion of the stimulating assembly 116. In FIG. 7A, line 733 represents the cochlear microphonic magnitude recorded from the primary recording site, i.e., the fixed target position at an angular insertion depth (AID) of approximately 90°. FIG. 7B illustrates the angular insertion depth of the most apical electrode contact throughout the insertion (line 735), as well as the angular insertion depth for other electrode contacts when they are the closest to the 90° target position. In FIG. 7B, the electrode number increases from the most apical electrode contact (i.e., electrode 1) to the most basal contact inserted into the cochlea 127, which in this case is electrode 19. For ease of illustration, the lines illustrating the angular insertion depth for electrode contacts 2-19 have not been individually labeled in FIG. 7B.

FIG. 7C is a zoomed in (enlarged) view of the cochlear microphonic magnitude (line 733 of FIG. 7A) at the fixed target position (90° AID). As shown, while there are some small fluctuations in magnitude that can be attributed to noise and small positional changes of the recording electrodes, the magnitude is steady and suggests that there was no disruption to acoustic hearing during this insertion and any drops in the cochlear microphonic magnitude observed on the apical electrode were due to local anatomy changes only.

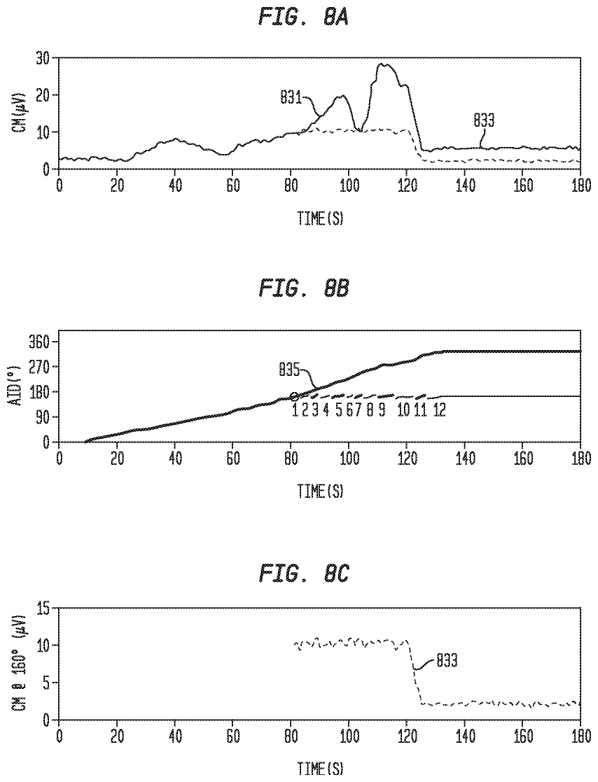

FIGS. 8A, 8B, and 8C schematically illustrate example monitored parameters of the ECochG signals obtained during another example insertion of stimulating assembly 116 into cochlea 127. However, whereas the target position for the primary recording site in FIGS. 6A-6F was an angular insertion depth of approximately 45°, in the examples of FIGS. 7A-7C the target position for the primary recording site is approximately 160°.

More specifically, FIG. 8A includes trace/line 831 representing the cochlear microphonic (CM) amplitude/magnitude recorded from the secondary recording site, i.e., the most apical electrode contact. In FIG. 8A, line 833 represents the cochlear microphonic magnitude recorded from the primary recording site, i.e., the target fixed location at an angular insertion depth (AID) of approximately 160°. FIG. 8B illustrates the angular insertion depth of the most apical electrode contact throughout the insertion (line 835), as well as angular insertion depth for other electrode contacts when they are the closest to the 160° target position for monitoring the cochlear microphonic magnitude. In FIG. 8B, the electrode number increases from the most apical electrode contact (i.e., electrode 1) to the most basal contact inserted into the cochlea 127, which in this case is electrode 12. For ease of illustration, the lines illustrating the angular insertion depth for electrode contacts 2-12 have not been individually labeled in FIG. 8B.

FIG. 8C is a zoomed in (enlarged) view of the cochlear microphonic magnitude (line 833 of FIG. 8A) at the fixed target position (160° AID). As shown, the small fluctuations in the cochlear microphonic magnitude can be attributed to noise and small positional changes of the recording electrodes. In addition, the cochlear microphonic magnitude at the fixed target position is steady until around the 120 seconds time point where there is a large drop in the cochlear microphonic magnitude recorded at both the apical electrode and the fixed target position. This suggests that there is a significant disruption to acoustic hearing during the insertion at about 120 seconds, whereas the earlier drop at 100 seconds in the cochlear microphonic magnitude from the most apical electrode contact (line 141 in FIG. 8A) was due to local cochlear anatomy.

In the examples of FIGS. 8A-8C, upon detecting significant disruption to acoustic hearing, the ECochG insertion monitoring system 180 generates an output to initiate a corrective action. For example, the ECochG insertion monitoring system 180 could issue a notification or alert (e.g., audible alert, visual alert, tactile alert, etc.) to the surgeon or other user. In addition or alternatively, the ECochG insertion monitoring system 180 could provide a recommendation to the surgeon based on the ECochG signals and position information (e.g., recommend to halt insertion, recommend to retract stimulating assembly by 45°). In the case of robotic or automated insertion system, the ECochG insertion monitoring system 180 could be integrated as part of the control sub-system and cause the robotic or automated insertion to execute the recommended action (e.g., halt insertion, retract stimulating assembly by 45°). In certain embodiments, feedback relating to the raw metric of the change (which may or may not have exceeded a threshold) can be provided. For example, this feedback can indicate the degree to which the CM magnitude has deviated from the reference response at the fixed target position. Such feedback could be provided via, for example, continuous visual/audio/tactile feedback (e.g., a number that is updated on a screen).

As noted, FIGS. 6A-6F, 7A-7C, and 8A-8C have generally been described with reference to relative analysis of multiple ECochG signals recorded from different sites, in combination with position information, to monitor the acoustic responsiveness of the inner ear. It is to be appreciated that the relative analysis of multiple ECochG signals has been used above merely to facilitate a complete understanding of the techniques presented herein, and that aspects of the techniques presented herein could be implemented using ECochG signals recorded from a single site, in combination with position information, to monitor the acoustic responsiveness of the inner ear.

For example, expanding on the embodiments of FIGS. 8A-8C, aspects of the techniques presented herein could only record, or only monitor the cochlear microphonic magnitude recorded from the primary recording site (e.g., only record, or only monitor signal 833, while not recording, or at least not monitoring, signal 831). Stated differently, such embodiments would operate similar to as described above, but potentially not record signals from the secondary recording site, or record signals from the secondary recording site, but not analyze/monitor the signals from the secondary recording site. Instead, the system would monitor to detect a drop in the cochlear microphonic magnitude 833 recorded from the single fixed inner ear location (i.e., drop the fixed electrode at the primary recoding site). A drop in the cochlear microphonic magnitude 833 could be used to determine a change in the acoustic responsiveness of the inner ear.

As noted, FIGS. 6A-6F, 7A-7C, and 8A-8C illustrate techniques in which the secondary recording site is fixed at the apical electrode and the primary recording site is a fixed position (e.g., angular insertion depth) relative to the cochlea 127 (e.g., the electrode used at the primary recording site changes over time, while the position of the primary recording site remains substantially fixed/constant). In accordance with other embodiments, the secondary recording site is fixed at the apical electrode and the primary recording site is a fixed at a different electrode, meaning that the position of the primary recording site relative to the cochlea changes over time.

More specifically, in accordance these embodiments, the acoustic stimulus 187 (e.g., 500 Hertz (Hz) tone pip) is delivered to the cochlea 127 and the recording module 188 records at least a few milliseconds of the voltage traces of the acoustic evoked response (ECochG signal) at the secondary recording site (i.e., the most apical electrode contact) and the second recording site (i.e., a different electrode). As noted, the secondary recording site and the primary recording site are fixed with respect to the stimulating assembly 116 and move with respect to the cochlea 127 as the stimulating assembly is advanced into the cochlea.

As the stimulating assembly 116 is inserted, the ECochG insertion monitoring system 180 continuously estimates the position of the stimulating assembly 116 (e.g., the position of the secondary recording site and the primary recording site) relative to the cochlea. For example, the ECochG insertion monitoring system 180 could determine the angular insertion depth for every electrode contact inside the cochlea at least 2× per second, and optionally also determine the position of the electrode contacts within the cross-sectional plane of the cochlear turn at each angle, or in the cylindrical coordinate system, or other 3-dimensional coordinate system). The ECochG insertion monitoring system 180 logs the acoustic evoked responses and the positions corresponding to each site when they were recorded.

In these examples, the ECochG insertion monitoring system 180 operates by comparing the magnitudes of acoustic evoked at nearby locations across time. Nearby locations can be, for example, Euclidean distances less than or equal to half the distance of the electrode spacing (e.g. 0.375 mm). If the difference, or ratio, of the acoustic evoked responses for a location decreases from the maximum response at that location by more than a threshold, for example 30% smaller, then determine that there is a significant drop in response at that location.

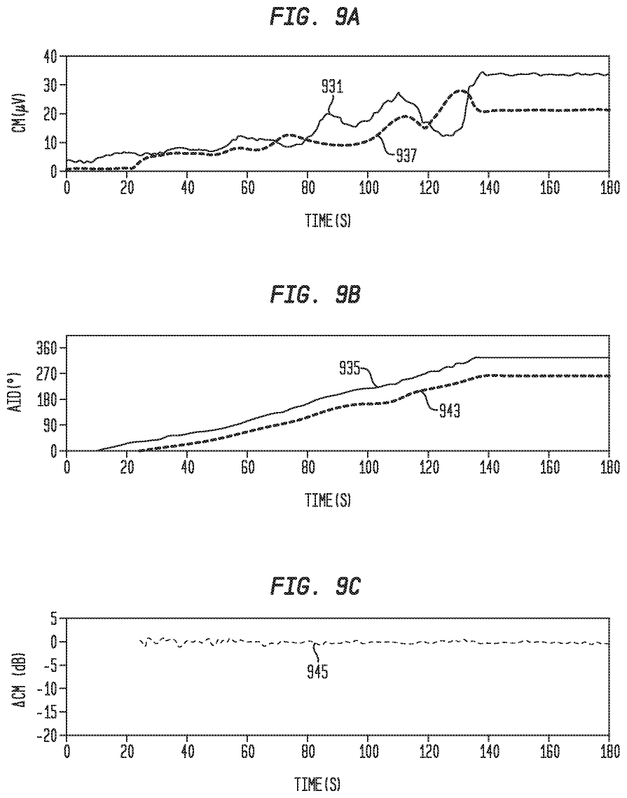

FIGS. 9A, 9B, and 9C schematically illustrate example monitored parameters of the ECochG signals obtained during an example insertion of stimulating assembly 116 into cochlea 127 where the secondary recording site and primary recording site are each fixed to an electrode contact. More specifically, FIG. 9A includes trace/line 931 representing the cochlear microphonic (CM) amplitude/magnitude recorded from the secondary recording site, i.e., the most apical electrode contact and a line 937 representing the cochlear microphonic magnitude from a second electrode contact located 3 mm more basal from the most apical contact.

FIG. 9B includes line 935 representing the angular insertion depth (AID) of the most apical electrode contact throughout the insertion, as well as line 943 representing the angular insertion depth of the other electrode contact corresponding to the primary recording site. FIG. 9C includes line 945 representing the ratio change, in decibels (dB), of the cochlear microphonic magnitude recorded from the more basal electrode contact (primary recording site) to the cochlear microphonic magnitude previously recorded from the most apical contact at the same cochlear position (e.g., substantially same insertion depth). Note that, in these examples, the cochlear microphonic ratio is steady and suggests that there was no disruption to acoustic hearing during this insertion and any drops in the cochlear microphonic magnitude observed on the apical electrode were due to local anatomy.

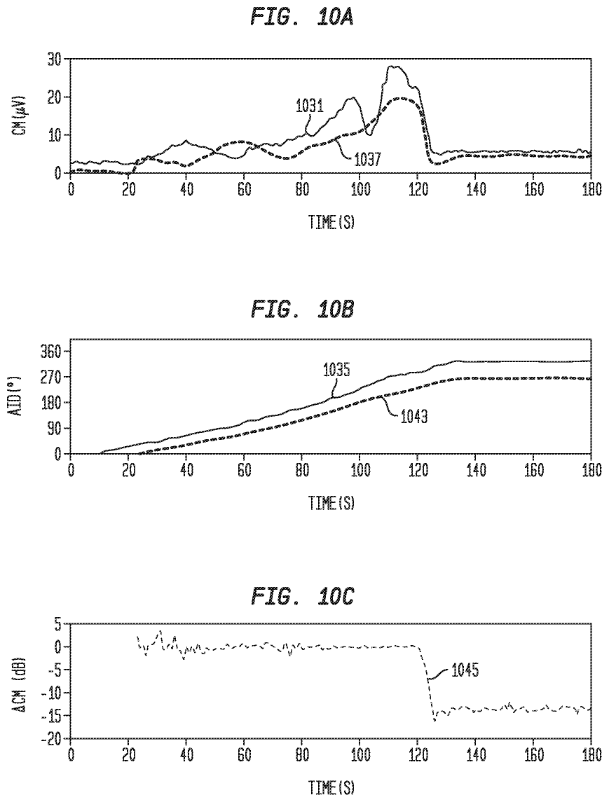

FIGS. 10A, 10B, and 10C schematically illustrate example monitored parameters of the ECochG signals obtained during another example insertion of stimulating assembly 116 into cochlea 127 where the secondary recording site and primary recording site are each fixed to an electrode contact. More specifically, FIG. 10A includes trace/line 1031 representing the cochlear microphonic (CM) amplitude/magnitude recorded from the secondary recording site, i.e., the most apical electrode contact and a line 1037 representing the cochlear microphonic magnitude from a second electrode contact located 3 mm more basal from the most apical contact.

FIG. 10B includes line 1035 representing the angular insertion depth (AID) of the most apical electrode contact throughout the insertion, as well as line 1043 representing the angular insertion depth of the other electrode contact corresponding to the primary recording site. FIG. 10C includes line 1045 representing the ratio change, in dB, of the cochlear microphonic magnitude recorded from the more basal electrode contact (primary recording site) to the cochlear microphonic magnitude previously recorded from the most apical contact at the same cochlear location. In these examples, the cochlear microphonic ratio is steady until around time 120 seconds, where there is a sudden drop in the cochlear microphonic ratio. This suggests that there is a significant disruption to acoustic hearing during the insertion at about 120 seconds, whereas the earlier drop at 100 seconds in the cochlear microphonic magnitude from the most apical electrode contact (line 1031, FIG. 10A) was due to local cochlear anatomy. In the examples of FIGS. 10A-10C, upon detecting significant disruption to acoustic hearing, the ECochG insertion monitoring system 180 generates an output to initiate a corrective action (e.g., issue a notification or alert, provide a recommendation to the surgeon, etc.).

As noted above, when a recording of the ECochG signal is generated, the position of the electrode contacts within the cochlea is estimated and associated with the recorded ECochG signal. In order words, recordings of an ECochG signal from either the secondary recording site or a primary recording site will include: (1) the ECochG signal that is recorded (e.g., the cochlear microphonic, the cochlear summating potential, and the auditory nerve neurophonic, etc. measured independently or in various combinations) and (2) the estimated position of the electrode contact when the ECochG signal was recorded. The ECochG signal recorded from the cochlea and the estimated electrode position are collectively referred to as the “ECochG signal data.” In certain embodiments, each recording of an ECochG signal could also include timing information (e.g., timestamp) for when an ECochG signal that is recorded, and this timing information could be included in the ECochG signal data.

A number of different techniques could be used to estimate/determine a position of an electrode contact. For example, a position of an electrode contact (e.g., angular insertion depth) could be estimated based on visual tracking (surgical microscope and/or visual markers on the stimulating assembly 116), video of insertion site, fluoroscopy or other imaging modality, based on data captured during robotic insertion or another insertion with a sensor for tracking length of stimulating assembly inserted into the cochlea, and/or derived from electrical measurements obtained from the stimulating assembly 116 (e.g. changes to electrical impedance as the electrode contacts are moved from a non-conductive medium, such as air, into a conductive medium, such as perilymph). The one or more electrical measurements can include one or more impedance measurements, such as a four-point impedance measurement. A four-point impedance measurement can involve passing a current between two electrode contacts and measuring an impedance induced between an inner pair of electrode contacts. Other impedance measurements can include a transimpedance measurement, a two-point impedance measurement, etc. In certain examples, the one or more electrical measurements can collect ECochG signals or neural responses and uses this data (e.g., analysis of the phase/latency of the ECochG signal, to, for example, determining best frequency or characteristic frequency for recording, electrode contact position, etc.

In certain examples, if the position of one electrode is determined, the system can estimate the position of the other electrode contacts. For example, in certain such embodiments, the techniques can use a plurality of intraoperatively collected electrical measurements, such as impedance measurements (e.g., CG, MP1, MP2 and MP12), transimpedance matrices (TIMs), and intracochlear bipolar impedances, in combination with intraoperative CT imaging or postoperative CT imaging to identify relationships of electrical measurements and/or features thereof to determine position of electrode contacts inside of the cochlea. For example, relationships of electrical measurements and/or features thereof can be used in combination with models and/or algorithms (e.g., statistical models, probabilistic models, first principle models, etc.) to estimate the real-time position of the electrodes of a stimulating assembly inside of the cochlea of a recipient. In these embodiments, electrical measurements can be taken, processed, and input into the models and/or algorithm to estimates of the position of the electrodes. As used herein, the position of the electrode contacts can include the position relative to the modiolus (e.g., modiolar proximity), angle of insertion, insert distance from the mid-modiolar axis, or other information relating to the placement or position of one or more parts of an electrode array. Further details regarding techniques for estimating electrode contact position are described in U.S. Provisional Application No. 63/277,253, filed Nov. 9, 2021, the content of which is hereby incorporated by reference herein.

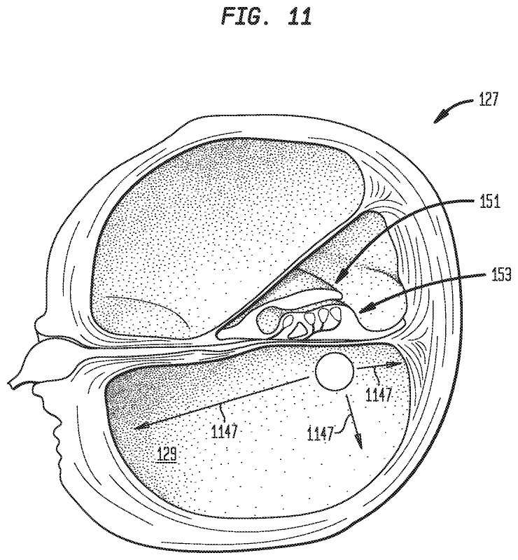

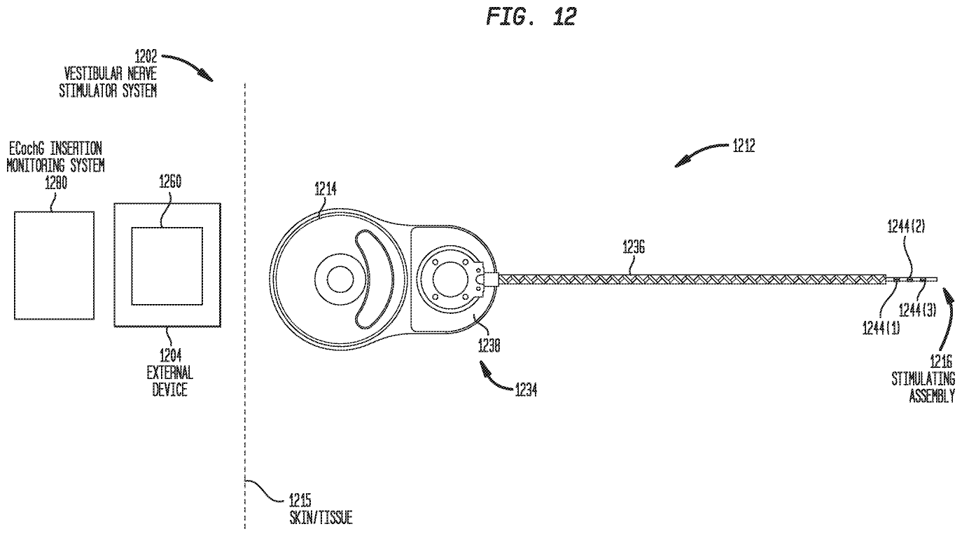

As noted, position of the electrodes can include the position relative to the modiolus (e.g., modiolar proximity), angle of insertion, distance from the mid-modiolar axis, or other information relating to the placement or position of one or more parts of an electrode array. FIG. 11 is a cross-sectional view of cochlea 127 with stimulating assembly 127 in the Scala tympani 129. In this cross-section, the stimulating assembly 127 is very close to the organ of Corti 151 and the outer hair cells (OHCs) 153 and, as a result, the cochlear microphonic magnitude would be its largest for this section of the cochlea (at a particular angle/depth). If the stimulating assembly 127 is further away from this position in the same section, either later, medial, or inferior (as indicated by the three arrows 1147), the cochlear microphonic magnitude is expected to decrease with distance from the OHCs. In accordance with the techniques presented herein, knowledge of the three-dimensional position of the electrode contacts with the Scala tympani can be used to compensate, or correct, for these different positions.

The above examples have been described with reference to use of one acoustic stimuli during the insertion process. In certain embodiments, multiple different acoustic stimuli having, for example, different frequencies could also be used during the insertion process. For example, in embodiments using a primary recording site with a fixed position relative to the cochlea, the system can record ECochG signals evoked by both an acoustic stimulus associated with a nominal target frequency (e.g., fixed low frequency such as 500 Hz) and a frequency that is optimal for the fixed position (based on an estimate or first determined best response frequency at the fixed position). In embodiments in which the secondary recording site and primary recording site are each fixed at electrode contacts, the system can record ECochG signals evoked by both an acoustic stimulus associated with a nominal target frequency (e.g., fixed low frequency such as 500 Hz) and a second frequency that changes/varies according to insertion depth. For example, the second frequency can be a frequency that changes, over time, so as to be a frequency associated with a tonotopic place between the two recording electrodes. The techniques could record ECochG signals evoked by one or more further acoustic stimuli, such as an acoustic stimulus having a changing frequency so as to remain just basal to the primary recording site.

As noted above, the techniques presented herein can provide surgeons or other users with an understanding of surgically recorded ECochG signals and the implications for surgeons. That is, the ECochG insertion monitoring system 180 is configured to automatically analyze the ECochG signal data and provide surgeons or other users with guidance as to when to perform a surgical intervention to maximize/balance hearing preservation and electrode insertion depth (e.g., provide surgeons with information to guide their decision process and maximize preservation of residual hearing and overall outcomes).

In one example, the ECochG insertion monitoring system 180 is configured such that an alarm/alert is generated when the system detects a significant disruption to acoustic hearing, but no alarm when the system determines a cochlear microphonic magnitude change due to local anatomy. In specific examples, the system does not sound an alarm if the stimulating assembly is retracted to a point that before had a lower cochlear microphonic magnitude. Similarly, if the surgeon withdraws the stimulating assembly to recover from a drop and goes past the location of the peak response, the system takes into consideration the new location when determining if the drop has recovered.

As previously described, the technology disclosed herein can be applied in any of a variety of circumstances and with a variety of different devices. For example, FIG. 12 illustrates an example vestibular stimulator with which aspects of the techniques presented herein can be implemented. The techniques of the present disclosure can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. These different systems and devices can benefit from the technology described herein.

As noted, FIG. 12 illustrates an example vestibular stimulator system 1202, with which embodiments presented herein can be implemented. As shown, the vestibular stimulator system 1202 comprises an implantable component (vestibular stimulator) 1212 and an external device/component 1204 (e.g., external processing device, battery charger, remote control, etc.). The external device 1204 comprises a transceiver unit 1260. As such, the external device 1204 is configured to transfer data (and potentially power) to the vestibular stimulator 1212. Also shown is an ECochG insertion monitoring system 1280.

The vestibular stimulator 1212 comprises an implant body (main module) 1234, a lead region 1236, and a stimulating assembly 1216, all configured to be implanted under the skin/tissue (tissue) 1215 of the recipient. The implant body 1234 generally comprises a hermetically-sealed housing 1238 in which RF interface circuitry, one or more rechargeable batteries, one or more processors, and a stimulator unit are disposed. The implant body 134 also includes an internal/implantable coil 1214 that is generally external to the housing 1238, but which is connected to the transceiver via a hermetic feedthrough (not shown).

The stimulating assembly 1216 comprises a plurality of electrode contacts 1244(1)-(3) disposed in a carrier member (e.g., a flexible silicone body). In this specific example, the stimulating assembly 1216 comprises three (3) stimulation electrode contacts, referred to as stimulation electrode contacts 1244(1), 1244(2), and 1244(3). The stimulation electrode contacts 1244(1), 1244(2), and 1244(3) function as an electrical interface for delivery of electrical stimulation signals to the recipient's vestibular system.

The stimulating assembly 1216 is configured such that a surgeon can implant the stimulating assembly adjacent the recipient's otolith organs via, for example, the recipient's oval window. It is to be appreciated that this specific embodiment with three stimulation electrode contacts is merely illustrative and that the techniques presented herein can be used with stimulating assemblies having different numbers of stimulation electrode contacts, stimulating assemblies having different lengths, etc.

As noted, shown in FIG. 12 is an ECochG insertion monitoring system 1280. The ECochG insertion monitoring system 1280 can be configured to implement the above described techniques to monitor insertion of the stimulating assembly 1216 into the recipient's inner ear (e.g., obtain and analyze ECochG signal data during insertion of the stimulating assembly 1216). It is to be noted that, in the embodiments of FIG. 12, the vestibular system may not be stimulated based on acoustic sounds, but instead stimulating based on, for example, low-frequency vibrations, motion data (e.g., linear or rotational acceleration signals), or other signals that can evoke an ECochG signal from the vestibular system.

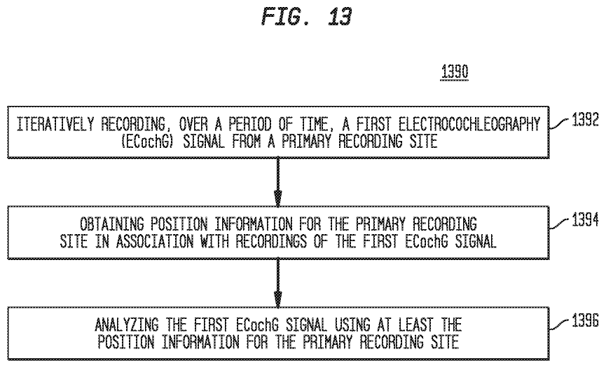

FIG. 13 is a flowchart of an example method 1390 for insertion of a stimulating assembly comprising a plurality of electrode contacts into an inner ear of a recipient, in accordance with certain embodiments presented herein. Method 1390 begins at 1392 where an ECochG insertion monitoring system (e.g., ECochG insertion monitoring system 180, 1280, etc.) iteratively records, over a period of time, a first ECochG signal from a primary recording site. At 1394, the ECochG insertion monitoring system obtains position information for the primary recording site in association with recordings of the first ECochG signal. At 1396, the ECochG insertion monitoring system analyzes the first ECochG signal using the position information for the primary recording site.

In certain embodiments, the method 1390 of FIG. 13 further includes iteratively recording, over the period of time, a second ECochG signal from a secondary recording site and obtaining position information for the secondary recording site in association with recordings of the second ECochG signal. In such examples, the first ECochG signal is analyzed relative to the second ECochG signal using the position information for each of the primary recording site and the secondary recording site.

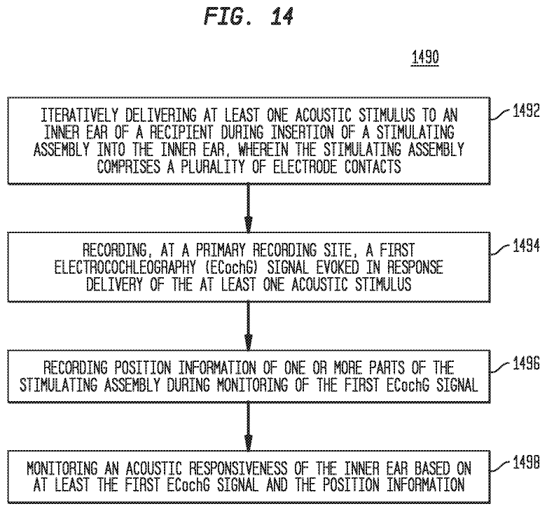

FIG. 14 is a flowchart of a method 1490, in accordance with certain embodiments presented herein. Method 1490 begins at 1492 where an ECochG insertion monitoring system (e.g., ECochG insertion monitoring system 180, 1280, etc.) iteratively delivers at least one acoustic stimulus to an inner ear of a recipient during insertion of a stimulating assembly into the inner ear, wherein the stimulating assembly comprises a plurality of electrode contacts. At 1494, the ECochG insertion monitoring system records, at a primary recording site, a first Electrocochleography (ECochG) signal evoked in response delivery of the at least one acoustic stimulus. At 1496, the ECochG insertion monitoring system records position information of one or more parts of the stimulating assembly during monitoring of the first ECochG signal. At 1498, the ECochG insertion monitoring system monitors an acoustic responsiveness of the inner ear based on at least the first ECochG signal and the position information.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments can be combined with another in any of a number of different manners.

1. A method for insertion of a stimulating assembly comprising a plurality of electrode contacts into an inner ear of a recipient, comprising:
iteratively recording, over a period of time, a first Electrocochleography (ECochG) signal from a primary recording site;
obtaining position information for the primary recording site in association with recordings of the first ECochG signal; and
analyzing the first ECochG signal using at least the position information for the primary recording site.

2. The method of claim 1, further comprising:
iteratively recording, over the period of time, a second ECochG signal from a secondary recording site;
obtaining position information for the secondary recording site in association with recordings of the second ECochG signal; and
analyzing the first ECochG signal relative to the second ECochG signal using the position information for each of the primary recording site and the secondary recording site.

3. The method of claim 2, wherein iteratively recording, over the period of time, the second ECochG signal from the secondary recording site comprises:
iteratively recording the second ECochG signal via a first electrode at a plurality of different positions within the inner ear,
wherein the secondary recording site is fixed at the first electrode.

4. The method of claim 3, wherein iteratively recording the second ECochG signal via the first electrode at the plurality of different positions within the inner ear comprises:
iteratively recording the second ECochG signal via a most apical electrode contact of the plurality of electrode contacts at the plurality of different positions within the inner ear.

5. The method of claim 4, wherein iteratively recording, over the period of time, the first ECochG signal from the primary recording site comprises:
iteratively recording the first ECochG signal at a substantially fixed target position within the inner ear via a series of different electrode contacts of the plurality of electrode contacts.

6. The method of claim 5, wherein analyzing the first ECochG signal relative to the second ECochG signal using the position information for each of the primary recording site and at least one secondary recording site comprises:
detecting a drop in a parameter of the second ECochG signal recorded from the first electrode; and
determining whether the drop in the parameter of the second ECochG signal is accompanied by a corresponding drop in a same parameter of the first ECochG signal recorded at the substantially fixed target position via one or more of the series of different electrode contacts.

7. The method of claim 6, further comprising:
determining whether the drop in the parameter of the second ECochG signal and the corresponding drop in a same parameter of the first ECochG signal recorded at the substantially fixed target position each exceed a predetermined threshold.

8. The method of claim 6, further comprising:
determining that a drop in the parameter of the second ECochG signal is accompanied by a corresponding drop in a same parameter of the first ECochG signal recorded at the substantially fixed target position via one or more of the series of different electrode contacts; and
in response to determining that a drop in the parameter of the second ECochG signal is accompanied by a corresponding drop in a same parameter of the first ECochG signal, generating an output to initiate a corrective action.

9. The method of claim 1, wherein analyzing the first ECochG signal using at least the position information for each of the primary recording sites comprises:
monitoring the first ECochG signal for a predetermined change in a parameter of the first ECochG signal.

10. The method of claim 9, wherein monitoring the first ECochG signal for a predetermined change in a parameter of the first ECochG signal comprises:
monitoring the parameter of the first ECochG signal for a change that exceeds a predetermined threshold.

11. The method of claim 2, wherein iteratively recording, over the period of time, the first ECochG signal from the primary recording site comprises:
iteratively recording the first ECochG signal via a second electrode at a plurality of different positions within the inner ear,
wherein the primary recording site is fixed at the second electrode.

12. The method of claim 11, wherein analyzing the second ECochG signal relative to the first ECochG signal using the position information for each of the secondary recording site and at least one primary recording site comprises:
generating a reference value relating a parameter of the second ECochG signal to a parameter of the first ECochG signal over the period of time; and
monitoring the reference value to detect when the reference value drops by an amount exceeding a predetermined threshold value.

13. The method of claim 12, further comprising:
determining that the reference value drops by an amount exceeding the predetermined threshold value; and
in response to determining that the reference value drops by an amount exceeding the predetermined threshold value, generating an output to initiate a corrective action.

14. The method of claim 12, wherein generating a reference value relating the parameter of the second ECochG signal to the parameter of the first ECochG signal over the period of time comprises:
computing a ratio of the parameter of the second ECochG signal to the parameter of the first ECochG signal over the period of time.

15. The method of claim 2, further comprising:
obtaining timing information for recordings of the second ECochG signal and recordings of the first ECochG signal.

16. The method of claim 15, further comprising:
analyzing the second ECochG signal relative to the first ECochG signal using the position information for each of the secondary recording site and at least one primary recording site and the timing information.

17. The method of claim 2, wherein obtaining position information for the secondary recording site and the primary recording site in association with recordings of the second ECochG signal and recordings of the first ECochG signal, respectively comprises:
estimating an angular insertion depth of one or more of the plurality of electrodes when recording the second ECochG signal or the first ECochG signal, or
estimating a proximity of one or more of the plurality of electrodes to a wall of the inner ear when recording the second ECochG signal or the first ECochG signal.

18. (canceled)

19. The method of claim 2, wherein analyzing the second ECochG signal relative to the first ECochG signal using the position information for each of the secondary recording site and at least one primary recording site, comprises:
analyzing an attribute of a cochlear microphonic associated with the second ECochG signal relative to an attribute of a cochlear microphonic associated with the first ECochG signal.

20. (canceled)

21. The method of claim 2, wherein analyzing the second ECochG signal relative to the first ECochG signal using the position information for each of the secondary recording site and at least one primary recording site, comprises:
analyzing an attribute of a cochlear summating potential associated with the second ECochG signal relative to an attribute of a cochlear summating potential associated with the first ECochG signal, or
analyzing an attribute of an auditory nerve neurophonic associated with the second ECochG signal relative to an attribute of an auditory nerve neurophonic associated with the first ECochG signal.

22. (canceled)

23. The method of claim 2, wherein analyzing the second ECochG signal relative to the first ECochG signal using the position information for each of the secondary recording site and at least one primary recording site, comprises:
determining based on the second ECochG signal, the first ECochG signal, and the position information, a change in acoustic responsiveness of the cochlea; and
in response to determining change in the acoustic responsiveness of the cochlea, generating an output to initiate a corrective action.

24. The method of claim 23, wherein generating an output to initiate a corrective action comprises:
generating at least one of an audible, visible, or tactile alert, or
generating a recommendation of the corrective action to initiate.

25. The method of claim 23, wherein generating an output to initiate a corrective action comprises:
determining the corrective action to initiate; and
generating a control signal representing the corrective action.

26-52. (canceled)

53. One or more non-transitory computer readable storage media comprising instructions that, when executed by a processor, cause the processor to:
obtain a second Electrocochleography (ECochG) signal iteratively recorded via an apical electrode of a stimulating assembly during insertion of the stimulating assembly into an inner ear of a recipient;
obtain a first ECochG signal iteratively recorded via at least one other electrode of the stimulating assembly during insertion of the stimulating assembly into the inner ear; and
analyze the second ECochG signal recorded via the apical electrode relative to the first ECochG signal recorded via at least one other electrode to characterize an acoustic responsiveness of the inner ear.

54. The one or more non-transitory computer readable storage media of claim 53, wherein the instructions that, when executed by the processor, cause the processor to analyze the second ECochG signal relative to the first ECochG signal to characterize an acoustic responsiveness of the inner ear comprise instructions that, when executed, cause the processor to:
determine whether a change to the second ECochG signal is due to local anatomy variations as the stimulating assembly moves through the inner ear, or whether the change to the second ECochG signal is due to a change in the acoustic responsiveness of the inner ear.

55. The one or more non-transitory computer readable storage media of claim 53, further comprising instructions that, when executed by the processor, cause the processor to:
obtain position information recorded for of one or more parts of the stimulating assembly during recording of the first ECochG signal and the second ECochG signal; and
characterize the acoustic responsiveness of the inner ear further based on the position information.

56. The one or more non-transitory computer readable storage media of claim 53, wherein the instructions that, when executed by the processor, cause the processor to analyze the second ECochG signal relative to the first ECochG signal to characterize an acoustic responsiveness of the inner ear comprise instructions that, when executed, cause the processor to:
detect a drop in a parameter of the second ECochG signal recorded from the apical electrode; and
determining whether the drop in the parameter of the second ECochG signal is accompanied by a corresponding drop in a same parameter of the first ECochG signal.

57. The one or more non-transitory computer readable storage media of claim 56, further comprising instructions that, when executed by the processor, cause the processor to:
determine whether the drop in the parameter of the second ECochG signal and the corresponding drop in a same parameter of the first ECochG signal each exceed a predetermined threshold.

58. The one or more non-transitory computer readable storage media of claim 53, wherein the instructions that, when executed by the processor, cause the processor to analyze the second ECochG signal relative to the first ECochG signal to characterize an acoustic responsiveness of the inner ear comprise instructions that, when executed, cause the processor to:
generate a reference value relating a parameter of the second ECochG signal to a parameter of the first ECochG signal; and
monitor the reference value to detect when the reference value drops by an amount exceeding a predetermined threshold value.

59. The one or more non-transitory computer readable storage media of claim 58, further comprising instructions that, when executed by the processor, cause the processor to:
determine that the reference value drops by an amount exceeding the predetermined threshold value; and
in response to determining that the reference value drops by an amount exceeding the predetermined threshold value, generate an output to initiate a corrective action.

60. The one or more non-transitory computer readable storage media of claim 53 55, further comprising instructions that, when executed by the processor, cause the processor to:
obtain timing information for recording of the second ECochG signal and recordings of the first ECochG signal; and
analyzing the second ECochG signal relative to the first ECochG signal using the timing information.

61. The one or more non-transitory computer readable storage media of claim 53, further comprising instructions that, when executed by the processor, cause the processor to:
determine a change in acoustic responsiveness of the inner ear; and
in response to determining change in the acoustic responsiveness of the inner ear, generate an output to initiate a corrective action.

62. (canceled)

63. (canceled)

64. (canceled)

65. A system, comprising:
a user interface;
a network interface for communication with an implantable medical device comprising a plurality of electrode contacts configured to be implanted into a recipient;
a memory; and
one or more processors configured to:
obtain recordings of a first Electrocochleography (ECochG) signal from a primary recording site during implantation of the plurality of electrode contacts into the recipient;
determine position information for the primary recording site in association with the recordings of the first ECochG signal; and
analyze the recordings of the first ECochG signal using at least the position information.

66. The system of claim 65, wherein the one or more processors are configured to:
obtain recordings of a second ECochG signal from a secondary recording site during implantation of the plurality of electrode contacts into the recipient;
determine position information for the secondary recording site in association with the recordings of the second ECochG signal; and
analyze the first ECochG signal relative to the second ECochG signal using the position information for each of the primary recording site and the secondary recording site.

67. The system of claim 66, wherein to determine position information for the secondary recording site and the primary recording site in association with recordings of the second ECochG signal and recordings of the first ECochG signal, respectively, the one or more processors are configured to:
estimate an angular insertion depth of one or more of the plurality of electrodes when recording the second ECochG signal or the first ECochG signal, or
estimate a proximity of one or more of the plurality of electrodes to a wall of the inner ear when recording the second ECochG signal or the first ECochG signal.

68. (canceled)

69. (canceled)

70. (canceled)

71. (canceled)
